(19) United States
(12) Patent Application Publication
(10) Pub. No.: US 2025/0113981 A1
(43) Pub. Date: Apr. 10, 2025

(54) ENDOSCOPIC CAMERA SYSTEM WITH ADJUSTABLE FIELD OF VIEW

(71) Applicant: Patrick A. MASSEY, Shreveport, LA (US)

(72) Inventor: Patrick A. MASSEY, Shreveport, LA (US)

(21) Appl. No.: 18/906,309

(22) Filed: Oct. 4, 2024

Related U.S. Application Data
(60) Provisional application No. 63/542,432, filed on Oct. 4, 2023.

(51) Int. Cl. A61B 1/00; A61B 1/04; A61B 1/05

(52) U.S. Cl. CPC A61B 1/00183; A61B 1/000095; A61B 1/00042; A61B 1/00045; A61B 1/00096; A61B 1/042; A61B 1/05

(57) ABSTRACT

An endoscopic camera system with adjustable field of view includes an endoscopic cable, a camera, a lens disposed at a distal end of the endoscopic cable, a view control, an image processor, and a display. Characteristics of a field of view of the camera are adjusted based on at least one input of the view control. An image of the field of view of the camera is processed by the image processor to generate a processed image, the processed image is displayed on the display, and the image is processed based on the characteristics of the field of view so as to modify proportions and/or orientation of the image such that the processed image displayed on the display has correct proportions and orientation.

### CROSS-REFERENCE TO RELATED APPLICATIONS

This application claims priority to U.S. Provisional Application 63/542,432, filed Oct. 4, 2023, the entire contents of which are hereby incorporated by reference.

### BACKGROUND

Endoscopic or arthroscopic cameras are used for surgery and mechanical inspections. There are two types of endoscopes, which differ based on the location of the camera. A rear-camera endoscope utilizes a camera that attaches to a proximal end of the endoscope and that has an eyepiece that views an image through the flexible tube of the endoscope. The lens at the distal end of the flexible tube is angled, allowing the user to view portions of the subject area disposed at various angles in relation to the distal end of the flexible tube, by rotating the tube. A light source is also provided at the distal end of the tube, typically connected to the tube at a side thereof. By rotating the scope and lens, while maintaining the camera at a horizontal orientation, the user can rotate the angle of view, with the image also being maintained in a horizontal orientation. A front-camera endoscope, in turn, has the camera disposed at the distal end of the tube.

A rear-camera endoscope presents a problem that, in order to view the subject area at different angles, the scope must be disconnected from the camera, a new scope, with a lens having a different angle, must be attached. The user must therefore have several different-angled scopes at their disposal. Additionally, switching the scope is time-consuming and requires withdrawal of the scope from the subject area. A front-camera endoscope presents a different problem, in that when the scope and tube with camera are rotated, the image that is output rotates as well. The change in orientation of the image can make image interpretation difficult.

### SUMMARY

According to at least one exemplary embodiment, an endoscopic camera system with adjustable field of view includes an endoscopic cable, a camera, a lens disposed at a distal end of the endoscopic cable, a view control, an image processor, and a display. Characteristics of a field of view of the camera may be adjusted based on at least one input of the view control. An image of the field of view of the camera may be processed by the image processor to generate a processed image, the processed image may be displayed on the display, and the image may be processed based on the characteristics of the field of view so as to modify proportions and/or orientation of the image such that the processed image displayed on the display has correct proportions and orientation.

In some exemplary embodiments, the lens may be a fisheye lens, and the endoscopic camera system may be a front-camera system or a rear-camera system. The field of view of the camera may correspond to a full field of view of the fisheye lens and the image is processed to generate a virtual field of view that is a partial field of view of the full field of view. The image may be further processed to remove lens distortion from the image, and the position and/or size of the virtual field of view may be adjustable based on the at least one input of the view control.

In some exemplary embodiments, the endoscopic camera system is a front-camera system, and the position of the camera is adjustable based on the at least one input of the view control, thereby adjusting the position of the field of view of the camera. The field of view of the camera is offset at an angle to a longitudinal axis of the endoscopic cable, and may be rotatable about the longitudinal axis of the endoscopic cable. The inclination of the field of view of the camera with respect to the longitudinal axis of the endoscopic cable may adjustable.

According to some exemplary embodiments, a method for processing and displaying a image of an endoscopic camera system is disclosed. The method can include receiving a viewed image from a camera of the endoscopic camera system, processing the viewed image, by an image processor, so as to generate a processed image, and displaying the processed image on a display. The viewed image may be processed based on characteristics of a field of view of the camera so as to modify proportions and/or orientation of the image such that the processed image displayed on the display has correct proportions and orientation. The characteristics of the field of view of the camera may be adjusted based on a type of lens of the camera and based on at least one input of a view control of the endoscopic camera system.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary embodiment of an endoscopic camera system with adjustable field of view.

FIG. 2A shows the embodiment of FIG. 1 with a full field of view and an adjustable partial field of view in a first exemplary position.

FIG. 2B shows the embodiment of FIG. 1 with a full field of view and an adjustable partial field of view in a second exemplary position.

FIG. 2C shows the embodiment of FIG. 1 with a full field of view and an adjustable partial field of view in a third exemplary position.

FIG. 3 shows another exemplary embodiment of an endoscopic camera system with adjustable field of view.

FIG. 4 shows another exemplary embodiment of an endoscopic camera system with adjustable field of view.

FIG. 5A shows the embodiment of FIG. 4 with an adjustable field of view in a first exemplary position.

FIG. 5B shows the embodiment of FIG. 4 with an adjustable field of view in a second exemplary position.

FIG. 5C shows the embodiment of FIG. 4 with an adjustable field of view in a third exemplary position.

FIG. 6A shows an exemplary embodiment of a rotary position sensor or rotary encoder.

FIG. 6B shows another view of the exemplary embodiment of a rotary position sensor or rotary encoder.

FIG. 6C another view of the exemplary embodiment of a rotary position sensor or rotary encoder.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

According to at least one exemplary embodiment, an endoscopic camera system with an adjustable field of view is disclosed. The endoscopic camera system may be provided as a front-camera endoscopic system or a rear-camera endoscopic system.

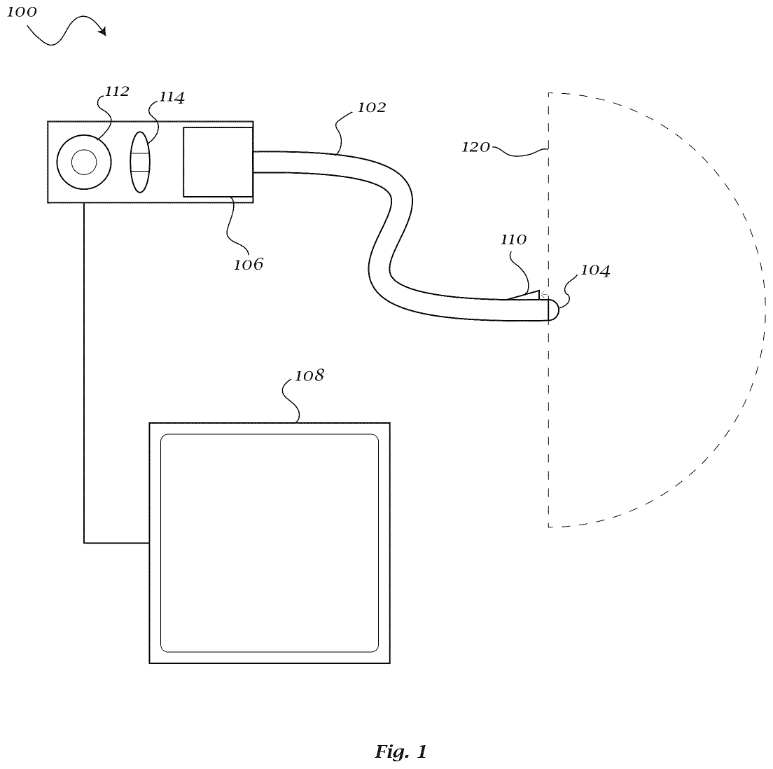

FIG. 1 shows an exemplary embodiment of a rear-camera endoscopic system 100. The rear-camera endoscopic system can include an endoscopic cable 102, a wide-angle objective lens 104 mounted at a distal end of the endoscopic cable, and a camera 106 mounted at the proximal end of the endoscopic cable and receiving an image from the wide-angle objective lens. The wide-angle objective lens 104 may have a field of view of 180° or substantially similar thereto, generally in the range of 160-180°. The wide-angle lens 104 may be, for example, a fisheye lens, or any type of lens or lens arrangement that enables system 100 to function as described herein. Camera 106 may be a camera or image sensor as generally known in the art and suitable for the applications described herein. The image viewed by camera 106 may be output to a display 108. Endoscopic cable 102 may likewise be any type of endoscopic cable that enables system 100 to function as described herein.

The rear-camera endoscopic system 100 may further include a light source 110, a rotation control 112, and additional controls 114. The rotation control 112 may be provided on a handpiece 116 of system 100. In some exemplary embodiments, the rotation control may be a rotary position sensor/encoder which may detect a rotation of the handpiece, or a portion of the handpiece. Embodiments of the rotation sensor may be implemented by various structures. For example, the rotation sensor/encoder can include a rotating disc having a pair of pins at a position thereon, and a complementary stationary disc that includes a plurality of stationary pins around the circumference thereof. Contact between the pair of pins and one of the stationary pins can determine the rotation position of the endoscope. In other embodiments, Hall sensors, magneto-resistive sensors, resolvers, incremental or absolute optical encoders, as well as solid-state non-contact position sensors may be used. In yet other embodiments, the rotation control may be any type of rotation control and sensor that enables system 100 to function as described herein. Rotation of the endoscope, or of the distal end of the endoscope and lens 104 may also be provided according to known solutions that enable system 100 to function as described herein.

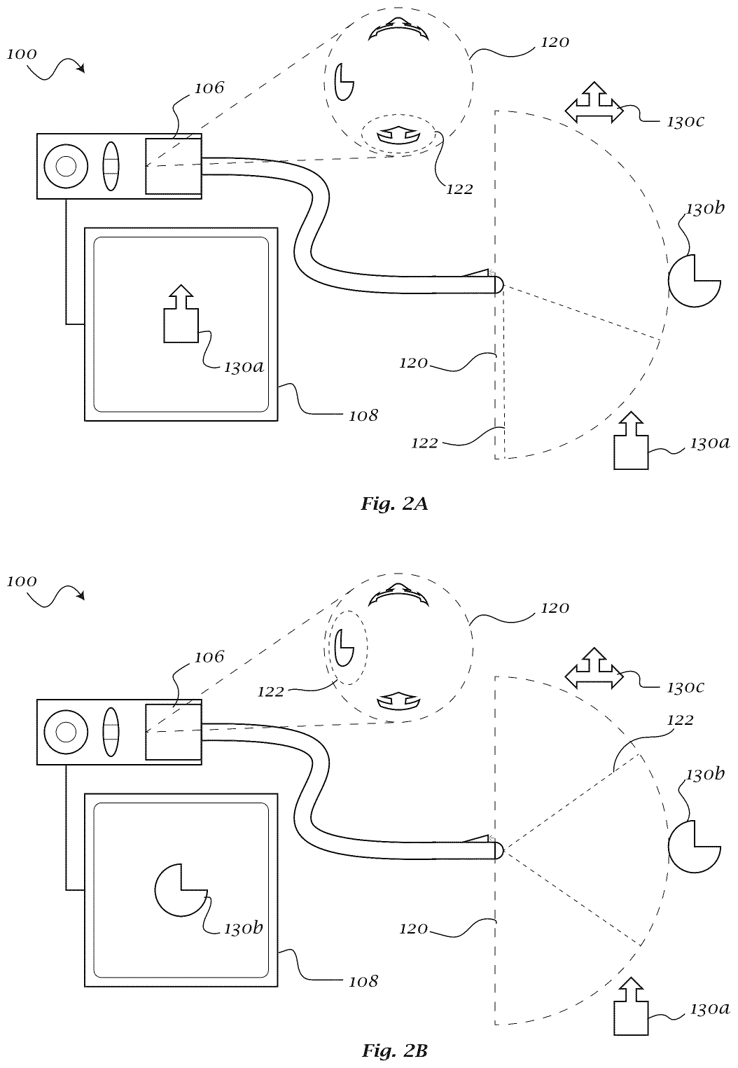

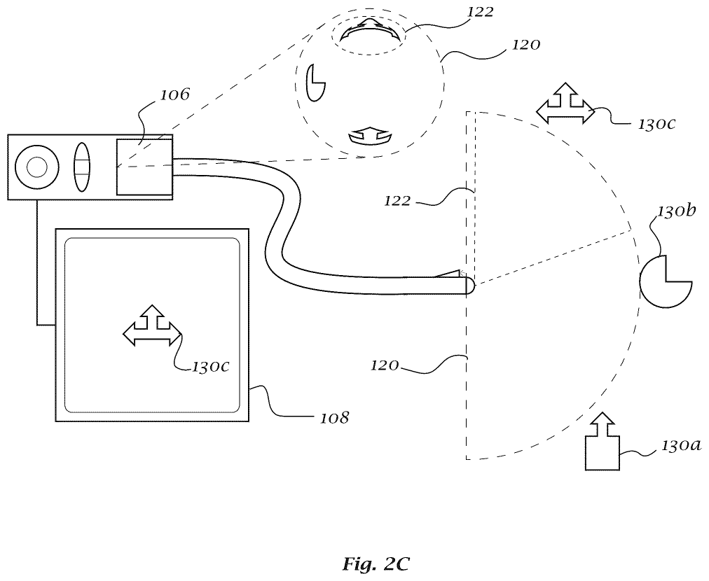

FIGS. 2A-2C show a diagrammatic two-dimensional representation of an exemplary full field of view 120 of the fisheye lens, and a virtual, partial field of view 122. The field of view may be approximately 180°. Objects 130 may be positioned within the full field of view 120. The image seen by camera 106 via lens 104 can be output to display device 108. Real-time image processing can convert a portion of the circular field of view 120 to a rectangular configuration so as to be displayed on the display while also removing the fisheye lens effect.

A user may select a partial field of view 122 of the full field of view 120 to be displayed on display 108 as a virtual field of view. The virtual, partial field of view may be, for example 70° or 30°. The user may then orient the field of view as desired using the rotation control 112. For example, as shown in FIG. 2A, the virtual, partial field of view 122 is oriented downwards, and includes object 130a therein. By operation of rotation control 112, the virtual, partial field of view can be moved in a circular arc within the full field of view 120, for example to positions 122b, and 122c, so as to capture objects 130b, 130c therein, as shown in FIGS. 2B-2C.

The virtual, partial field of view and its inclination may be calculated in real-time and based on the selected size of the field of view and selected position of rotation control 112. The size of the field of view may be calculated using the formulas D_{Outputfov}=L_{screen}*q_{Outputfov}/q_{Lensfov} and r_{Outputfov}=D_{Outputfov}/2, where L_{screen} is the length of the display, D_{Outputfov} is the diameter of the output virtual field of view, r_{Outputfov}=is the radius of the output virtual field of view, q_{Outputfov} is the angle of the selected output virtual field of view, and q_{Lensfov} is the the maximum angle of the wide-angle lens. The inclination of the field of view may be calculated using the formula r_{cov}=(q_{IOV}/q_{Lensfov})*L_{screen}, where r_{cov} is the distance from the center of the full field of view of the lens to the center of the virtual, partial field of view, and q_{IOV} is theaAngle of inclination of the field of view. Furthermore, for a rotary rotation control such as a rotary encoder/sensor, the location of the field of view may be calculated based on the position of the rotary encoder/sensor using the formulas X=r_{cov}*sin θ_{rotatory} and Y=r_{cov}*θ_{rotatory}, where θ_{rotatory} is the number of degrees clockwise or counterclockwise that the rotatory encoder/sensor has been rotated, with the 12 o'clock position being 0 and counterclockwise rotation being decreasing θ, X is the scalar horizontal coordinate of the center of rotation of the field of view, and Y is the scalar vertical coordinate of the center of rotation of the field of view.

Additional controls 114 may allow for further customization of the virtual, partial field of view. Such additional controls may be provided as physical controls on a handle of the endoscopic device or as virtual controls on the display. A control may be provided to allow customization or changing of the inclination of the partial field of view by moving the virtual, partial field of view more peripherally or more centrally from within the original lens field of view. This may be accomplished by moving the center of the selected partial field of view towards or away from the center of the 180° field of view. For example, by converting 30° into scalar coordinates, the center of the selected circle can be moved 16.6% downward in correspondence with the percent of the inclination of view of the full field of view of the fisheye lens.

A control may be provided to allow increasing and decreasing the field of view by selecting a different size for the virtual, partial field of view. This may be accomplished by increasing or decreasing the diameter of the outputted field of view. For example, for a 60° partial field of view, the diameter of the virtual field of view would be 33.3% of the full field of view of a 180° fisheye lens.

Furthermore, when switching from a 30° to a 70° degree inclination of view, the partial field of view may rotate around a circle of larger diameter. This may be accomplished by moving the center of the selected virtual, partial field of view downward from the center of the 180° field of view. For example, by converting 70° into scalar coordinates, the center of the selected circle can be moved 38.9% downward in correspondence with the percent of the inclination of view of the full field of view of the fisheye lens.

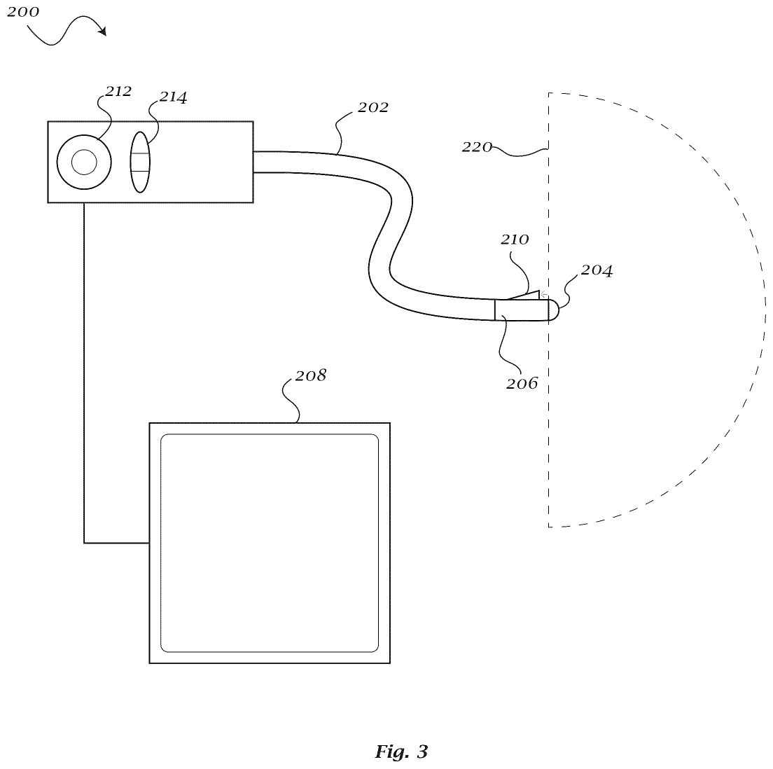

FIG. 3 shows an exemplary embodiment of a front-camera endoscopic system 200. In embodiment 200, similar components to embodiment 100 are labeled with similar reference numerals, but with a leading digit of 2. Embodiment 200 is substantially similar to embodiment 100, except that camera 206 is disposed at the distal end of cable 202, and is provided with a wide-angle objective lens 204, or disposed proximate wide-angle objective lens 204 so as to receive an image therefrom.

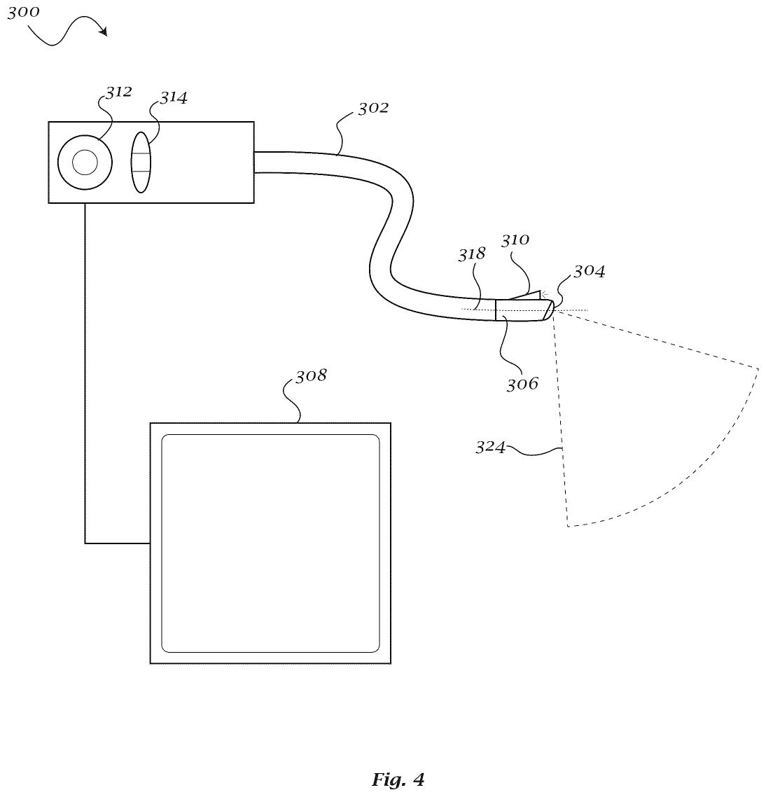

FIG. 4 shows an exemplary embodiment of a front-camera endoscopic system 300. The front-camera endoscopic system can include an endoscopic cable 302, and a camera 306 that is provided with an objective lens 304 mounted at a distal end of the endoscopic cable. Camera 306 may be a camera or image sensor as generally known in the art and suitable for the applications described herein. The image viewed by camera 306 may be output to a display 308. Endoscopic cable 302 may likewise be any type of endoscopic cable that enables system 300 to function as described herein.

As shown in FIG. 4, camera 306 may have a field of view 324 that is offset from the longitudinal axis 318 of cable 302. For example, field of view 324 of camera 306 may be offset from the longitudinal axis 318 of cable 302 by 30°, 45°, 60°, and so forth. Camera 306 may also be rotatably mounted within the distal end of endoscopic cable 302, such that camera 306 is rotatable about the longitudinal axis 318 of endoscopic cable 302.

The front-camera endoscopic system 300 may further include a light source 310, a rotation control 312, and additional controls 314. The rotation control 312 may be provided on a handpiece 316 of system 300. Rotation control 312 may be implemented substantially as in the description of rotation control 312 of system 300. User interaction with otation control 312 can result in the rotation of camera 306. In some exemplary embodiments, if rotation control is implemented as a rotatable wheel, camera 306 may be rotated by one degree for every one-degree rotation of rotation control 312. In other exemplary embodiments, various correspondences between rotation control 312 and the rotation of camera 306 may be contemplated and provided as desired.

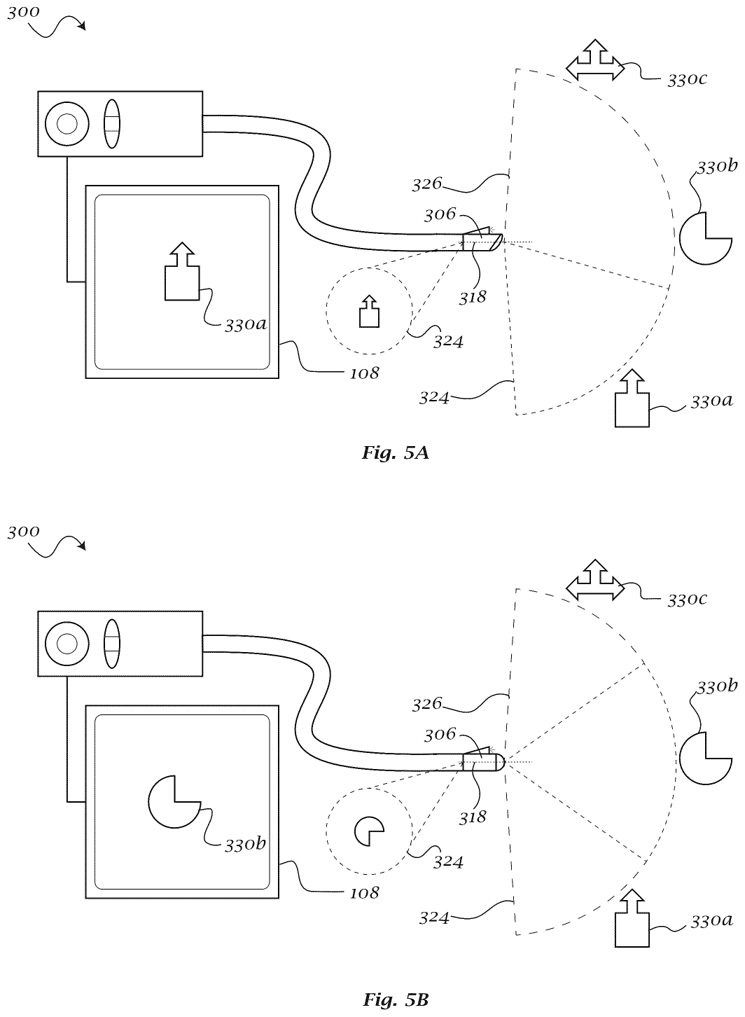

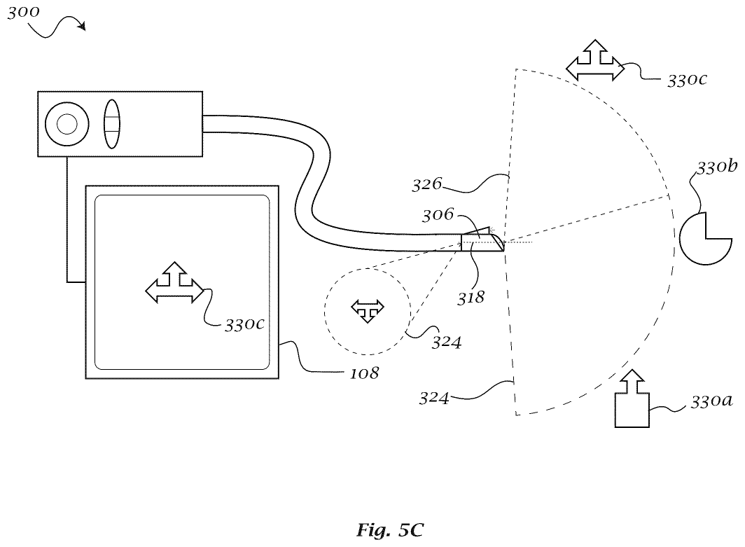

FIGS. 5A-5C show a diagrammatic two-dimensional representation of an exemplary potential field of view 326 of of camera 306. Objects 330 may be positioned within potential field of view 326; however, only objects disposed within the actual field of view 324 may be viewed via display 308. A user may rotate camera 306 by way of interaction with rotation control 312 so as to move the offset, actual field of view 324. For example, as shown in FIG. 5A, the offset, actual field of view 324a is oriented downwards, and includes object 330a therein. By operation of rotation control 312, the offset, actual field of view can be moved in a circular arc within the potential field of view 326, for example to positions 324b, and 324c, so as to capture objects 330b, 330c therein, as shown in FIGS. 5B-5C.

The image seen by camera 306 can be output to display device 308. It should be appreciated that as camera 306 is rotated, the orientation of the field of view is correspondingly rotated. Real-time image processing can convert the rotated field of view 324 to a maintain an upright orientation of the image displayed on display 308. Real-time image processing can further convert convert the field of view to a rectangular configuration so as to be displayed on display 308.

Additional controls 314 may allow for further customization of the offset, actual field of view. Such additional controls may be provided as physical controls on a handle of the endoscopic device or as virtual controls on the display. A control may be provided to allow changing of the inclination of the offset, actual field of view by moving the center of the field of view of the camera closer to or further away from the longitudinal axis of the endoscopic cable. A control may be provided to allow increasing and decreasing the field of view by selecting a different size for the virtual, partial field of view. This may be accomplished virtually, via image processing, or by adjusting a physical zoom factor of the lens of the camera, thereby increasing or decreasing the diameter of the outputted field of view.

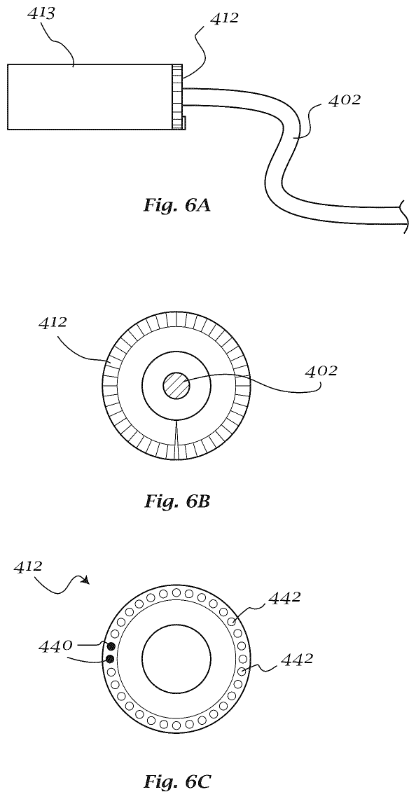

In exemplary embodiments of the endoscopic camera system, the rotation control may be implemented as a rotary position sensor or rotary encoder 412 disposed, for example, on a handpiece 413 of the endoscopic camera system. FIGS. 6A-6B show exemplary side and front views of a handpiece 413 with an exemplary rotary position sensor or rotary encoder 412 thereon. The rotary position sensor or rotary encoder may be rotated so as to rotate the field of view of the camera and/or the field of view that is shown on the display device. In some exemplary embodiments, the rotary position sensor can include an orientation knob, with the inclination of view of the camera being directed 180° opposite the knob. In some exemplary embodiments, rotary encoder can include a rotating disc disposed around endoscopic cable 402. The rotary encoder can include common pins 440 and stationary contact pins 442, as shown in FIG. 6C. In other exemplary embodiments, the rotation control may be implemented via magnetic hall-effect elements with digital output, magnetic hall-effect elements with analog output, a magneto-resitive sensor with analog output, a resolver, an incremental optical encoder, an absolute optical encoder, a solid-state fully sealed and completely non-contact position sensor, or any other implementation that enables the endoscopic camera system to function as described herein.

The rotation of the rotary control can control the rotation of the field of view of the camera and/or the field of view that is shown on the display device. For example a one-degree rotation of the rotary control can result in a one-degree rotation of the field of view of the camera. This may be implemented via actual rotation of the camera and its field of view and/or image processing of the image viewed by the camera. Additionally, image processing may be implemented to include horizontal leveling such that the field of view is maintained horizontal and/or upright regardless of the angle of the camera and/or the field of view of the camera.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An endoscopic camera system with adjustable field of view, comprising:
an endoscopic cable;
a camera;
a lens disposed at a distal end of the endoscopic cable;
a view control;
an image processor; and
a display;
wherein characteristics of a field of view of the camera are adjusted based on at least one input of the view control;
wherein an image of the field of view of the camera is processed by the image processor to generate a processed image;
wherein the processed image is displayed on the display; and
wherein the image is processed based on the characteristics of the field of view so as to modify proportions and/or orientation of the image such that the processed image displayed on the display has correct proportions and orientation.

2. The system of claim 1, wherein the lens is a fisheye lens and the endoscopic camera system is a rear-camera system or a front camera system.

3. The system of claim 2, wherein:
the field of view of the camera corresponds to a full field of view of the fisheye lens; and
the image is processed to generate a virtual field of view that is a partial field of view of the full field of view.

4. The system of claim 3, wherein:
the view control is a rotary control;
the virtual field of view is rotated one degree for every one-degree rotation of the rotary control; and
the image is further processed to maintain a same horizontal orientation of the processed image displayed on the display.

5. The system of claim 4, wherein the rotary control is a rotary position sensor or a rotary encoder.

6. The system of claim 3, wherein the image is further processed to remove lens distortion from the image.

7. The system of claim 6, wherein the position and/or size of the virtual field of view is adjustable based on the at least one input of the view control.

8. The system of claim 1, wherein:
the endoscopic camera system is a front-camera system; and
the position of the camera is adjustable based on the at least one input of the view control, by way of which the position of the field of view of the camera is adjusted.

9. The system of claim 8, wherein:
the field of view of the camera is offset at an angle to a longitudinal axis of the endoscopic cable;
the field of view of the camera is rotatable about the longitudinal axis of the endoscopic cable; and
the image is further processed as the field of view is rotated so as to maintain a same horizontal orientation of of the processed image displayed on the display.

10. The system of claim 9, wherein the view control is a rotary control and the virtual field of view is rotated one degree for every one-degree rotation of the rotary control.

11. The system of claim 9, wherein an inclination of the field of view of the camera with respect to the longitudinal axis of the endoscopic cable is adjustable.

12. A method for processing and displaying a image of an endoscopic camera system, comprising:
receiving a viewed image from a camera of the endoscopic camera system;
processing the viewed image, by an image processor, so as to generate a processed image; and
displaying the processed image on a display;
wherein the viewed image is processed based on characteristics of a field of view of the camera so as to modify proportions and/or orientation of the image such that the processed image displayed on the display has correct proportions and orientation; and
wherein the characteristics of the field of view of the camera are adjusted based on a type of lens of the camera and based on at least one input of a view control of the endoscopic camera system.

13. The method of claim 12, wherein the lens is a fisheye lens.

14. The method of claim 13, wherein:
the field of view of the camera corresponds to a full field of view of the fisheye lens; and
the image is processed to generate a virtual field of view that is a partial field of view of the full field of view.

15. The system of claim 14, wherein the image is further processed to:
maintain a same horizontal orientation of the processed image displayed on the display; and
remove lens distortion from the image.

16. The system of claim 15, wherein the position and/or size of the virtual field of view is adjustable based on the at least one input of the view control.

17. The system of claim 12, wherein:
the endoscopic camera system is a front-camera system; and
the position of the camera is adjustable based on the at least one input of the view control, thereby adjusting the position of the field of view of the camera.

18. The system of claim 17, wherein:
the field of view of the camera is offset at an angle to a longitudinal axis of the endoscopic cable;
the field of view of the camera is rotatable about the longitudinal axis of the endoscopic cable; and
the image is further processed as the field of view is rotated so as to maintain a same horizontal orientation of of the processed image displayed on the display.

19. The system of claim 18, wherein the view control is a rotary control and the virtual field of view is rotated one degree for every one-degree rotation of the rotary control.

20. The system of claim 18, wherein an inclination of the field of view of the camera with respect to the longitudinal axis of the endoscopic cable is adjustable.
